(19) United States
(12) Patent Application Publication
(10) Pub. No.: US 2019/0083074 A1
(43) Pub. Date: Mar. 21, 2019

(54) RIGID TONGUE RETRACTOR

(71) Applicants: Hari Reyes, Portland, OR (US); Keith Gressell, Parker, SD (US)

(72) Inventors: Hari Reyes, Portland, OR (US); Keith Gressell, Parker, SD (US)

(73) Assignee: LINGUAGUARD LLC, VANCOUVER, WA (US)

(21) Appl. No.: 16/191,997

(22) Filed: Nov. 15, 2018

Related U.S. Application Data
(63) Continuation of application No. 15/052,787, filed on Feb. 24, 2016.

(51) Int. Cl. A61B 13/00; A61C 17/06; A61B 90/00

(52) U.S. Cl. CPC A61B 13/00; A61B 2090/0817; A61C 17/043

(57) ABSTRACT

A dental device that firmly mates and rotationally orients the proximal end of a suction (evacuation) straw of a high volume extraction unit to a rigid, disposable tongue retractor firmly enough that it cannot be dislodged therefrom. This allows the device to direct the forcible movement and retention of the tongue while suctioning out saliva, water and debris from the dental patient's mouth. The device has a unique four point capture collar with two upper bands and two lower bands separated by open voids. The half bands each half encircle the shaft of the suction straw with the proximal void providing an easy connection of the straw's locking orifice to the retention post on the retractor.

### CLAIM FOR DOMESTIC PRIORITY

This application incorporates by reference and is a Continuation in Part of U.S. patent application Ser. No. 15/052,788 filed Feb. 24, 2016 and herein incorporates by reference all disclosed material and definitions therein.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to dentistry, and more particularly to oral cavity suction technology.

### BACKGROUND

When dental patients are being worked on, the procedures require the patients to recline at an angle and their ability to swallow is restricted. High volume suction devices are frequently used to either clean out debris or fluids from the mouth. Unfortunately, many patients are sedated or have frozen tongues making complete suction under and around the tongue, cumbersome if not problematic.

There are tools to allow the dentist or dental technician to position the tongue in the mouth using both a high volume evacuation device and having a secondary tool in the mouth, is clumsy and crowds the mouth. This often is just to allow momentary access to an area needing suction, while in other instances it is needed to force and restrain the tongue for extended periods in a position where the dentist can access the area they need.

A high volume evacuation device can “latch onto” the tongue because of its strong suction. This occurrence is abrupt, and scary to some patients. Furthermore, many high-volume evacuation devices are coupled at their operational end to a polymer straw with an angularly cut end that provides discomfort if it suddenly affixes itself to the tongue.

Henceforth, a rigid tongue retractor that allows for forceful positioning and retention of a human tongue from various different angles within the mouth and that can be connected to a suction straw so as to allow gentle suctioning of the patient's oral cavity, would be a well-received innovation in the dental industry.

### BRIEF SUMMARY

In accordance with various embodiments, a rigid apparatus for the application of force to move and constrain a human tongue is provided.

In one aspect, a rigid tongue retractor configured to accept a modified suction straw compatible with commercially available dental suction devices is provided.

In yet another aspect, a high-volume evacuation system is provided that is adapted for moving and securely holding the patient's tongue in a plethora of positions while still using a minimal of space available in the oral cavity. It provides a safeguard against the “latching on” of the device to the patient's tongue and it has a unique connection design that prevents the translational and rotational movement of the tongue retention paddle on the modified suction straw.

Various modifications and additions can be made to the embodiments discussed without departing from the scope of the invention. For example, while the embodiments described above refer to particular features, the scope of this invention also includes embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components.

FIG. 1 is a top perspective view of an embodiment of the rigid tongue retractor;

FIG. 2 is a bottom perspective view of an embodiment of the rigid tongue retractor;

FIG. 3 is a top view of the rigid tongue retractor;

FIG. 4 is a left-side view of the rigid tongue retractor;

FIG. 5 is a bottom view of the rigid tongue retractor;

FIG. 6 is a proximal end view of the rigid tongue retractor;

FIG. 7 is a right-side view of the rigid tongue retractor;

FIG. 8 is a distal end view of the rigid tongue retractor;

FIG. 9 is a top perspective view of the rigid tongue retractor coupled to a suction straw;

FIG. 10 is a top perspective of the suction straw;

FIG. 11 is a side view of the straw collar and tongue paddle axes; and

FIG. 12 is a top view of the straw collar and tongue paddle axes.

### DETAILED DESCRIPTION OF CERTAIN EMBODIMENTS

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention. It will be apparent to one skilled in the art, however, that other embodiments of the present invention may be practiced without some of these specific aspects and features.

Unless otherwise indicated, all numbers herein used to express quantities, dimensions, and so forth, should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

It will be understood that, although the terms first, second, etc. may be used herein to describe various elements, these elements should not be limited by these terms. These terms are only used to distinguish one element from another.

It will be understood that when an element is referred to as being “on,” “coupled to,” or “connected to” another element, it can be directly on, directly coupled to or directly connected to the other element, or intervening elements may be present. In contrast, when an element is referred to as being “directly on,” “directly coupled to,” or “directly connected to” another element, there are no intervening elements present.

The present invention relates to a novel design for a rigid, inflexible tongue retractor intended for use by attachment to a suction straw coupled to a high volume evacuation (suction) machine in a dental office. Extraction straws are inserted into the mouth to evacuate debris and fluids such as blood, saliva and water from a patient's mouth during dental procedures. Since these straws operate under high volume suction, they often quickly adhere to the inside of the patient's mouth, or the tongue. This is both alarming and painful to the patient, especially since the operative end of the suction straw comes to a point as it is usually cut on a bias with respect to its longitudinal axis. Commonly, the dental assistant will use a second tool to move and position the tongue away from the end of the suction straw, but this requires room in the patient's mouth. This is not preferred when the dentist is simultaneously working on the patient. The present invention, a rigid tongue retractor, is an adaptor that is fitted at the distal end of a modified suction straw and locked from non-rotational and non-translational movement when put under multi-directional forces.

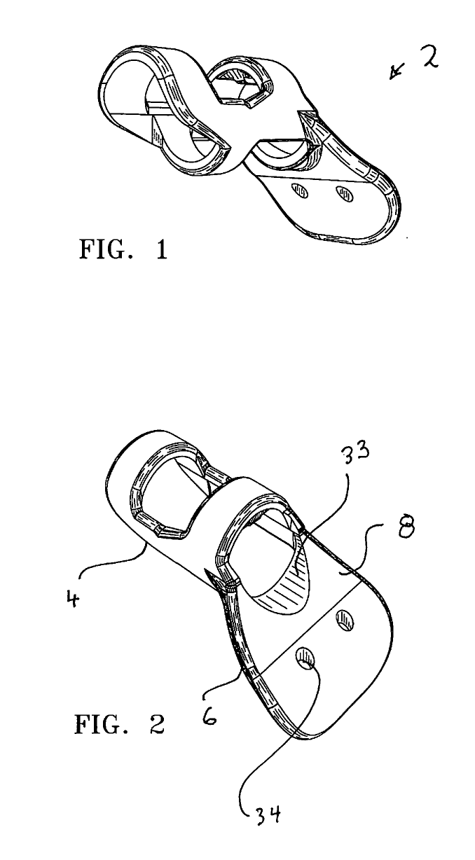

Looking at FIGS. 1 and 2 it can be seen that the tongue retractor 2 is a rigid unitary polymer body that has a generally cylindrical translational and rotational motion locking collar 4. The locking collar has a proximal end (the suction straw receiving end), a distal end and a first linear axis passing longitudinally through the interior of the locking collar 4. The distal end of the locking collar 4 has an S shaped tongue paddle 6 extending therefrom. (Best seen in the cross section of FIG. 7.) The tongue paddle 6 has a second linear axis that intersects the first linear axis of the locking collar 4 at an obtuse angle so that the two axes are not rectilinear.

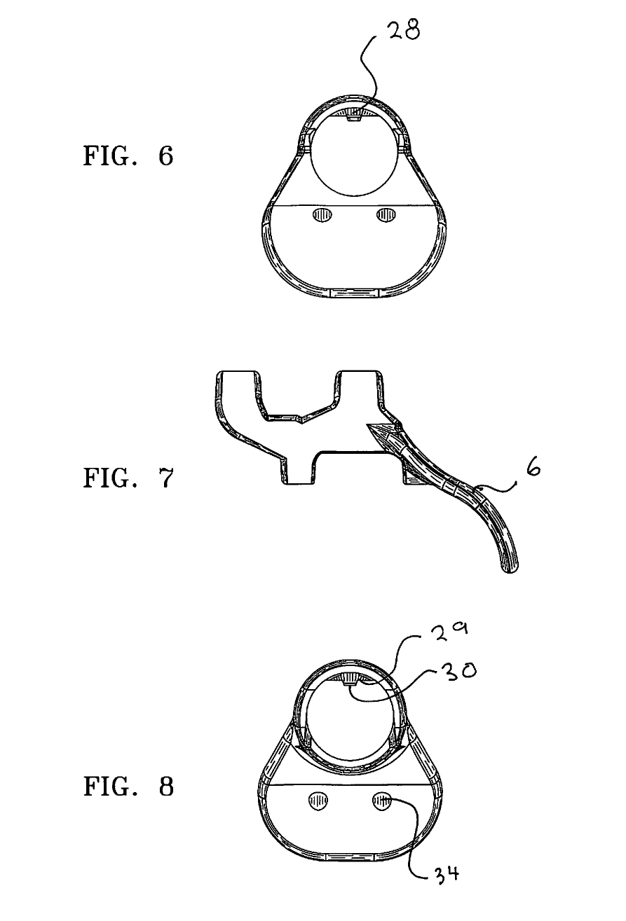

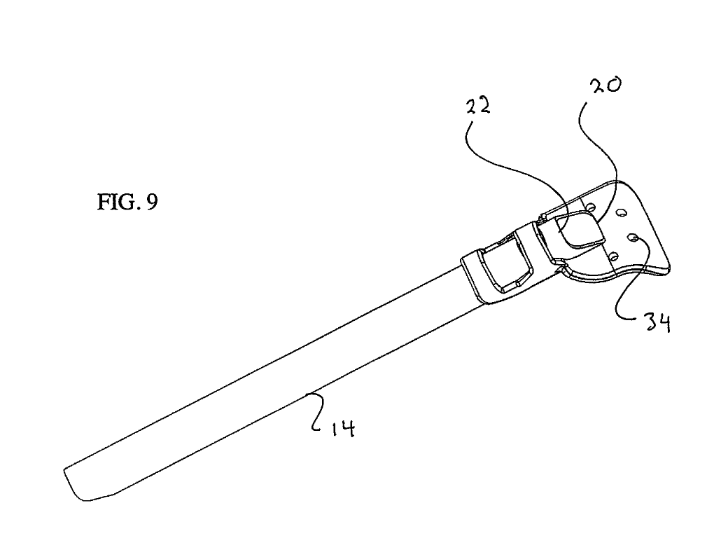

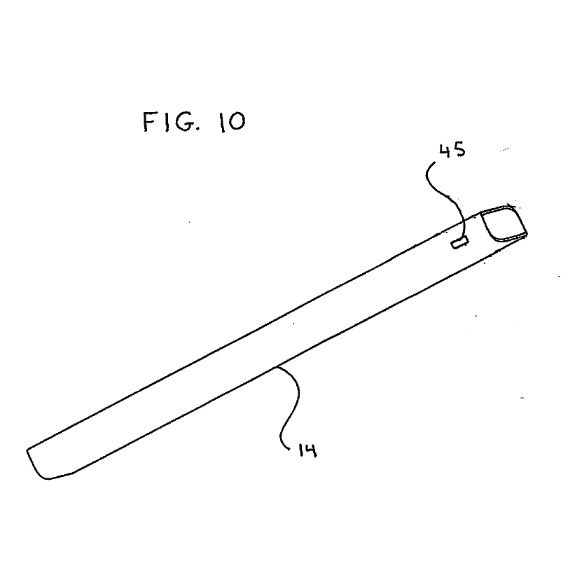

The locking collar 4 has a four-point support design that secures a suction straw 14 (FIG. 9) about four areas of its distal end, yet allows the distal end to extend past the locking collar's fourth support point and out onto the upper face 8 of the tongue paddle 6. This in conjunction with the locking tab 28 (FIGS. 5 and 6) restricts both translational and rotational movement between the suction straw 14 and the rigid tongue retractor 2. This is critical as the human tongue can develop up to 16 N of force and the tongue retractor 2 must be able to withstand this level of force without twisting or detaching from the suction straw 28.

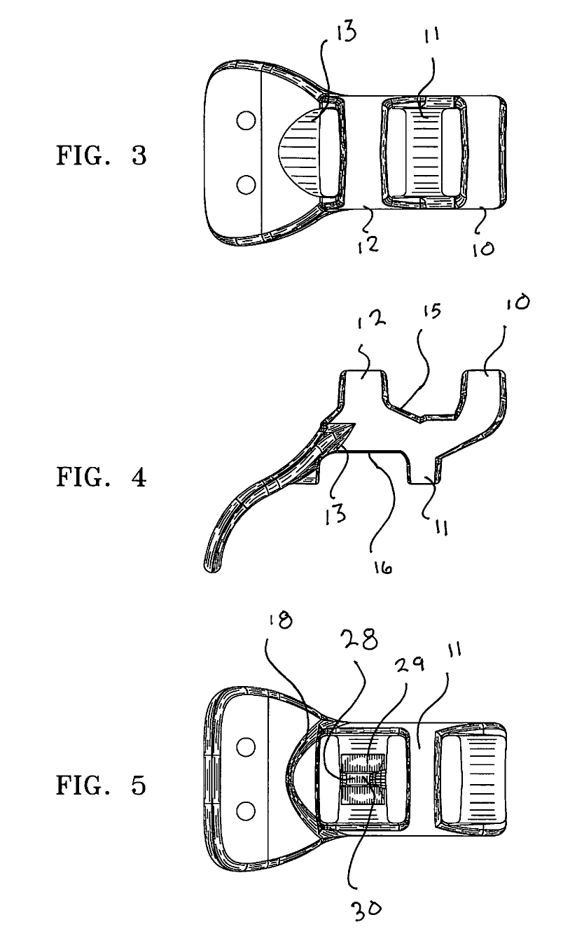

Looking at FIGS. 3-5, the four-point support system of the locking collar 4 has two upper semi-circular support bands 10 and 12 and two lower semi-circular support bands 11 and 13 The two pairs of support bands oppose each other but are offset. The first (most proximal) band 10 and third band 12 form the top half of the straw collar and partially encircle the upper half of an inserted suction straw 28, and the second band 11 and fourth (most distal) band 13 form the bottom half of the straw collar and partially encircle the lower half of an inserted suction straw 28. The first and third (upper) bands are separated by an upper transverse slot 15 and the second and fourth (lower) bands are separated by the lower transverse slot 16. These transverse slots are removed sections in the locking collar that extend across its width, perpendicularly to the linear axis of the straw collar 4. It is important to note that the lower transverse slot 16 has its edges 18 beveled or in the alternative, has a radius formed thereon. This is critical to the mating of a suction straw 28 to the tongue retractor 2. The duplicity of contact surfaces gives lateral stability to the assembled device.

As mentioned earlier, the pair of first and third upper bands and the second and fourth lower bands are offset with respect to each other. While they face each other, they are not directly oppositional. The lower transverse slot 16 lies directly below the third band 12 and the upper transverse slot 15 is above the second band 11.

Extending normally inward from the inside face of the third band is a rectangular translational and rotational locking tab 28. This locking tab matingly conforms to a locking orifice 45 cut through the side of the suction straw. This locking tab 28 is tapered around all of its sides, narrowing as it extends away from the third band's inner surface. This aids in the centering of the locking orifice 45 in the suction straw 14 onto the locking tab 28.

Through this design with the third band 12 directly above the lower transverse slot 13, the distal (bottom) end 20 of the suction straw 14 can be inserted at a slight downward angle into the distal end of the locking collar 4. Upon insertion of the suction straw 14 into the collar 4 the distal end of the suction straw 14 will dip into the lower transverse slot 16 so the top distal end 22 of the suction straw 14 will clear the locking tab 28. Since the distal end of the suction straw 14 is cut on an angle, its leading bottom end 20 will contact the radiused edge 18 on the fourth band 13 and it will momentarily deform at its bottom. As the suction straw 14 is inserted deeper into the tongue retractor 2 the radiused edge 18 on the fourth band 13 will direct and allow the suction straw 14 to pass beyond the straw collar 4 and onto the concave groove 40 on the top face 8 of the tongue paddle 6. As the angled distal end of the straw 14 moves deeper into the collar 4 over the radiused edge of the fourth band 13, the rigidity of the straw 14 increases reducing the bottom deformation and causing the straw 14 to rise in the collar 4 such that the orifice 45 in the straw 14 meets the beveled side faces of the transverse and rotational locking tab 28. Since the locking tab 28 is beveled on all of its side faces 29, its lower face 30 is smaller than the orifice 45 in the suction straw allowing for ease of alignment. The locking tab's upper perimeter matingly conforms to the internal size of the suction straw orifice 45. The tapered sides 30 of the locking tab 28 guide the mating engagement between the suction straw 14 and the locking collar 4. This type of engagement eliminates the necessity for having a slot to secure the suction straw to the tongue retractor as is common in the prior art. The slot type of engagement does not eliminate translational movement between the two elements as is encountered with puling motions.

This locking tab 28 and orifice in the suction straw 14 maintain the straw 14 and retractor 2 from translational and rotational movement allowing great forces to be put onto the paddle 6. The alignment also aligns the straw's cut end in the paddle correctly. When the locking tab 28 is inserted into the orifice in the suction straw 14, the angled tip 20 of the suction straw 14 is orientated so as to be supported in the concave groove 40 cut in the top face of the paddle beyond the distal end of the fourth band 13. After this engagement, the suction straw 14 is frictionally held and laterally supported by the four support bands 10-13 and the post 28 prevents linear separation and rotation of the suction straw from the tongue retractor.

The distal end of the S shaped tongue paddle 6 widens. The paddle 6 has a concave seat 40 formed along the top face of the paddle 6 beginning at the end of the fourth band 13 and matching the internal curvature of the straw collar 4. This seat 40 (FIG. 2) is centered longitudinally along the top face of the paddle 6. With matching curvatures, the distal, angularly cut suction end of the suction straw 14 (FIG. 9) passes completely through the straw collar 4, such that the suction straw resides with its distal end resting on the top face 8 of the paddle 6 in the concave seat 40.

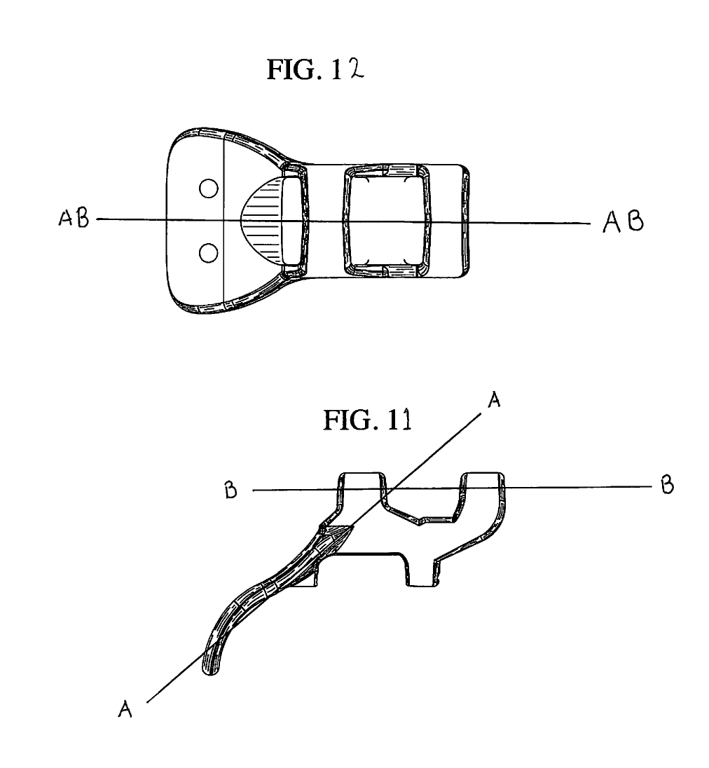

It is to be noted that the linear axis of the straw collar B and the linear axis of the paddle A intersect at an acute angle (FIG. 11) so as to form a straight line AB when viewed from the top (FIG. 12) although these two axes are not rectilinear.

The paddle 6 has an array of suction orifices 34 cut there through at an angle with respect to the top face 8 of the tongue paddle. Cutting these on an angle increases the size of the suction opening on the upper and lower faces of the paddle 6. This allows for more fluids and debris to be evacuated from both sides of the paddle then if the suction orifices 34 were perpendicular to the top and bottom faces of the paddle.

The distal end of the suction straw is cut on an angle so as to create an oval opening to prevent the suctioning of the tongue or cheek as is common with a circular opening.

Suction is only applied from the top face of the paddle 34. The peripheral edge of the paddle 34 is contoured or radiused to ensure it has no sharp edges and feels comfortable in a patient's mouth. The S shape of the paddle allows the dental assistant to push the tongue with the paddle's top face or pull and restrain the tongue with the paddle's bottom face. With the downward arch of the paddle the dental assistant can also lightly pull debris to the front of the mouth. The width of the paddle is ideal to gently urge the cheek away from the teeth and gums thereby allowing both room for the dentist to work and for the suctioning of the area between the two.

The inner diameter of the suction straw collar 4 is sized for mating frictional engagement about the outer diameter of a suction straw 14.

The tongue retractor is not intended for sterilization and reuse. It is a single use device. Its polymer material may vary however, it will be rigid and cost effective for injection molding.

While various embodiments are described with—or without—certain features for ease of description and to illustrate exemplary aspects of those embodiments, the various components and/or features described herein with respect to a particular embodiment can be substituted, added, and/or subtracted from among other described embodiments, unless the context dictates otherwise. Consequently, it will be appreciated that the invention is intended to cover all modifications and equivalents within the scope of the following claims.

Having thus described the invention, what is claimed as new and desired to be secured by Letters Patent is as follows:

1. A rigid tongue retractor for dental applications comprising;
a body having a distal end and a proximal end;
a four support point locking collar for a suction straw formed at said proximal end, said locking collar having a first linear axis;
a contoured tongue paddle formed at said distal end, said tongue paddle having a second linear axis;
a translational and rotational movement locking tab extending from an inside surface of said locking collar, said locking tab sized for insertion into a mating orifice formed at the distal end of said suction straw; and
wherein said locking collar and said tongue paddle are connected such said first linear axis and said second linear axis intersect at an angle;

2. The rigid tongue retractor of claim 1 further comprising:
a pair of upper semi-circular support bands forming a top half of said straw collar, said upper semi-circular support bands separated by an upper transverse slot extending across a width of said locking collar;
a pair of lower semi-circular support bands forming a bottom half of said straw collar, said lower semi-circular support bands separated by a lower transverse slot extending across a width of said locking collar;
wherein said upper semi-circular support bands oppose said lower semi-circular support bands in an offset configuration.

3. The rigid tongue retractor of claim 2 wherein said upper semi-circular support bands are made of a first support band at the proximal end of said suction straw collar and a third support band; and
wherein said lower semi-circular support bands are made of a second support band and a fourth support band at the distal end of said suction straw collar; and
wherein said second support band is directly below said upper transverse slot; and
wherein said third support band is directly above said lower transverse slot; and
wherein said locking tab is formed on an inner surface of said third support band.

4. The rigid tongue retractor of claim 3 further comprising an array of suction orifices with elliptical openings, said suction orifices extending through said tongue paddle at an angle with respect to a top face of said tongue paddle.

5. The rigid tongue retractor of claim 4 further comprising a concave seat for a distal end of said suction straw, said concave seat formed along said top face of said tongue paddle beginning at a distal end of said fourth support band.

6. The rigid tongue retractor of claim 5 wherein said translational and rotational movement locking tab has at least one tapered side narrowing toward the linear axis of said straw collar.

7. The rigid tongue retractor of claim 6 wherein said translational and rotational movement locking tab is rectangular with four inward tapered sides and wherein said orifice formed at the distal end of said suction straw is sized for mating engagement about a non-tapered portion of said locking tab.

8. The rigid tongue retractor of claim 7 wherein said straw has a distal end cut on an angle with respect to a linear axis of said suction straw.
